(19) United States
(12) Patent Application Publication
(10) Pub. No.: US 2019/0336255 A1
(43) Pub. Date: Nov. 7, 2019

(54) SINGLE-PIECE SUCTION MIRROR

(71) Applicant: CLEVERDENT LTD., Münster (DE)

(72) Inventors: Stephan CLASEN, Münster (DE); Martin KAYSER, Köln NRW (DE)

(21) Appl. No.: 16/474,310

(22) PCT Filed: Jan. 9, 2018
(86) PCT No.: PCT/EP2018/050478

§ 371 Date: Jun. 27, 2019

(30) Foreign Application Priority Data
Jan. 10, 2017 (DE) 10 2017 000 106.5

(51) Int. Cl. A61C 17/06; A61B 1/253; A61C 17/08

(52) U.S. Cl. CPC A61C 17/088; A61B 1/00094; A61C 17/08; A61B 1/253

(57) ABSTRACT

A dental suction mirror for suctioning liquids and particles from an oral cavity includes a tubular hollow main body having inner and outer surfaces, a longitudinal axis, a connection opening for a tube, and a suction opening. The inner surface has a mirror that can be observed at least in some regions through the suction opening, and has a mirror surface and a rear side. The main body is configured as a single piece and includes, in its inner surface in the suction opening region, a mirror receptacle configured as a recess and in which the mirror is held in a locking manner. A method for producing a suction mirror includes at least manufacturing a base body with a mirror-accommodating portion and inserting the mirror by pressing it into the mirror-accommodating portion such that an elastic upper retaining shoulder is pressed outwards and contacts an upper face thereof.

### TECHNICAL FIELD

The present disclosure relates to a dental mirror suction device for suctioning liquids and particles from an oral cavity of a patient, with a hollow base body having an outer surface, an inner surface, a longitudinal axis, a connection opening, and a suction opening, wherein the inner surface has a mirror viewable through the suction port and the base body is formed from a first base body part and a second base body part that are firmly connected to each other. The disclosure further relates to a method for producing such a mirror suction device.

### BACKGROUND

Dental treatments often require that emerging liquids or dissolved particles, such as saliva, spray water and blood, be suctioned off during treatment. Also, water, for example for cleaning or after using a multi-function syringe, may accrue, which has to be suctioned off. Usually, suction devices are used for this purpose, which are generally formed from a tubular body of plastic, to whose end a hose is attached, which in turn is connected to a pump. The distracting liquids and solids are carried away through the hose.

A suction device is often not guided and held by the attending dentist or dental surgeon himself but by an assistant, because the attending dentist has to hold a drilling tool with the one hand and a mirror with the other, with which he is able to inspect the area to be treated. The above-described procedure is disadvantageous in that the two people have to stand or sit very close to each other, around the area to be treated. Particularly if the interventions are rather difficult or demanding with regard to fine-motor skills, this may be perceived as distracting by the attending physician.

A medical mirror suction device in which the inner surface has a mirror-coated surface which is visible through the suction opening is known from DE 102006048463 A1. The reflective coating according to the disclosure enables the user to use the medical mirror suction device both as a mirror suction device for removing liquids and particles and, simultaneously, as a mirror. Using such a mirror suction device, it is now possible for them to carry out the treatment without an assisting person. Thus, the mirror suction device is simultaneously used as a suction device and as a mirror.

The basic concept of the combination is very good in principle; however, the practical implementation of the concept or the production of such a mirror suction device have proven extremely difficult. The mirror has to be firmly fitted; it must not become detached, and also should not be allowed to move during treatment because the view is affected thereby. In addition, no bumps or gaps may be present, if possible, because they have a negative effect on the development of noise.

For example, the base body of the mirror suction device may be formed from two longitudinal halves, which are welded together and retain the mirror in a groove. However, satisfactorily welding the longitudinal halves together in a gap-free manner is hardly possible; either a shadow gap is formed or excess material is produced during the welding process, which must then be removed. The shadow gap is unsightly, and liquids and bacteria may collect in it; the removal of the excess material is time-consuming and results in visible changes to the surface of the base body. Furthermore, the two longitudinal halves have to be extremely exactly manufactured in order to ensure an attractive, in particular gap-free and/or excess-free appearance after assembly or welding. They are troublesome particularly because the attending dentist feels any bump, groove or burr in the longitudinal extent of the mirror suction device with his fingers. In particular, only the smallest length tolerances of the two halves are acceptable in the longitudinal direction; other than that, the two halves must not be disposed with even a minimal offset relative to one another in the longitudinal direction.

### SUMMARY

The present disclosure provides a mirror suction device that has a mirror surface with a sufficiently high quality in the region of the suction opening. In this case, the mirror suction device is supposed to be cost-effective to produce and have an attractive exterior. The disadvantages of the prior art are to be avoided; in particular, the mirror suction device is supposed to have no, or only insubstantial, gaps on its outside. Further, the disclosure proposes a method for producing a mirror suction device with the aforementioned advantages.

According to the disclosure, a mirror suction device having the features of claim 1 achieves the aforementioned advantages. A production method according to the disclosure is specified in patent claim 7.

Accordingly, the mirror is formed from only a single base body. According to the disclosure, the base body is manufactured in a single process step, preferably by the injection-molding method.

A mirror-accommodating portion is configured as a depression or recess, into which the mirror is quickly and simply pressed from above in a second process step, subsequent to the production of the base body. Accordingly, the mirror-accommodating portion has an undercut formed by an upper retaining shoulder. Accordingly, the mirror-accommodating portion or the retaining shoulder is configured to be elastic, so that it is able to move back when the mirror is pressed in, and the mirror can be snapped into the mirror-accommodating portion.

In order to facilitate the insertion of the mirror, it is preferably configured to have a conical cross section. Starting from the mirror surface, its diameter decreases in the direction towards the back of the mirror.

After being inserted, the mirror is firmly secured in the mirror suction device, which is advantageous because the base body and the mirror can be treated as a unit in practical operation. The mirror and mirror suction device may be sterilized together. In this context, the term “firmly” means that the mirror is retained in a loss-proof manner; it cannot be detached without destroying the mirror suction device or the mirror.

An essential insight is that an attractive outward appearance of the base body is obtained when one of the two first base body parts is as large as possible and the other base body part is as small as possible and the smaller base body part extends in the longitudinal direction of the mirror suction device only to a small extent. Thus, the distracting groove or distracting burr produced by the connecting surfaces between the base body parts is relatively short.

Advantageously, the mirror is circular, but may also be oval or have other suitable shapes. The following is based on the customary circular shape of the mirror.

Advantageously, the material surrounding the mirror and retaining it is soft or elastic, so that it is able to compensate for expanded material as it may be produced during sterilization. Undesired tensions in the area of the mirror that are capable of destroying the mirror and/or the base body are thus effectively avoided. Alternatively or additionally, the mirror may be retained in a groove that is sufficiently deep in the lateral direction to compensate for thermal expansion or enlargement of the surface area and diameter of the mirror. The expanding mirror can extend into the sufficiently deep groove. Instead of the groove, the diameter of the opening in the first base body part, into which the mirror is inserted, may also have a slightly larger diameter than the mirror.

The mirror surface and the surrounding bottom surface form as planar an overall surface as possible, via which the air flow, suctioned-off liquid and particles can be carried away in an optimal manner. The planar overall surface also causes the noise development due to air turbulence in this region to be low. A protrusion of the mirror over the bottom surface of the first base body part of up to 0.3 mm is still considered to be flush in the sense of the disclosure.

Instead of a retaining shoulder extending around the entire outer circumference of the mirror, several, preferably three, uniformly distributed retaining shoulders may also be provided, which retain the mirror in its position. Thus, the overall surface is not planar, but in principle, such a configuration would work regardless.

A thermoplastic synthetic material, such as polypropylene or also polyethylene, is particularly suitable for production. By adding additives, the outward appearance of the mirror suction device may be influenced. A production using polyester has also proved to be advantageous according to the disclosure. With respect to the surface quality of the finished product, polyester has significant advantages over other plastics, e.g. with regard to the settable gloss level, scratch resistance and surface smoothness. Glass beads, which have an effect on, inter alia, the gloss level of the surface, are possible suitable aggregates for influencing the material properties.

The suction opening of the tubular base body does not extend at right angles to the longitudinal axis but is configured to be oblique relative to it. This results in an obliquely tapering shape of the mirror suction device, whereby it is easier to insert between the cheek and the teeth, for example. The mirror is not located in front of the suction opening, but substantially behind the suction opening in the flowing direction of the suctioned air, i.e. within the base body. It is thus achieved that the mirror suction device is not made longer by an upstream mirror, which would reduce the suctioning performance.

The method for producing the mirror suction device according to the disclosure comprises the following process steps:
manufacturing a base body with a mirror-accommodating portion, which is configured as a depression and has a substantially peripherally extending, elastic upper retaining shoulder, for the mirror,
inserting the mirror from above by pressing it into the mirror-accommodating portion in such a way that the upper retaining shoulder, due to its elasticity, is first pressed outwards by the mirror and then contacts an upper face of the mirror and retains the mirror in the mirror-accommodating portion.

### BRIEF DESCRIPTION OF THE DRAWINGS

The disclosure is explained further with reference to the following Figures. They merely show exemplary embodiments; the disclosure is not supposed to be limited thereto.

In the Figures:

FIG. 1: shows a mirror suction device according to the disclosure from above,

FIG. 2: shows the mirror suction device according to the disclosure of FIG. 1 from the side,

FIG. 3: shows the mirror suction device according to the disclosure of FIG. 1 in a longitudinal section, and

FIG. 4: shows an enlargement of the region A from FIG. 3.

### DETAILED DESCRIPTION OF THE DRAWINGS

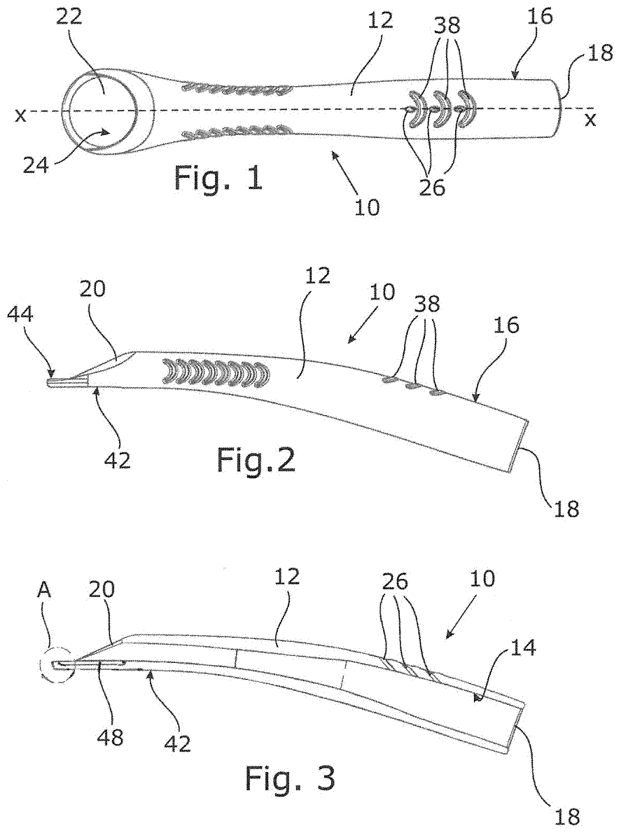

As is apparent, in particular, from FIGS. 1 to 3, a mirror suction device 10 according to the disclosure has a hollow, tubular base body 12 with an inner surface 14 and an outer surface 16. Moreover, the base body 12 has a longitudinal axis X-X (see FIG. 1). The arc shape of the mirror suction device 10, which is recognizable particularly in FIGS. 2 and 3, is advantageous in that the latter is easier to guide towards the site to be treated.

The base body 12 has a connection opening 18 for a hose, which is not shown, and a suction opening 20 for suctioning particles and liquids. The liquids or particles to be suctioned off are sucked through the connection opening 20 and carried away through the connection opening 18 via the hose.

According to the disclosure, a mirror 22, which is viewable at least in some portions through the suction opening 20, is disposed within the base body 12 in the region of the suction opening 20. Accordingly, a visible mirror surface 24 faces towards the suction opening 20. Preferably, the mirror 22 is disposed in its entirety within the base body 12, i.e. behind the suction opening 20 as viewed in the flowing direction of the air to be suctioned off; however, it may be disposed in some portions in front of the suction opening 20. The suctioned air is guided over the mirror surface 24, whereby fogging of the mirror surface 24 is effectively prevented.

The mirror suction device 10 has additional openings 26 through which air is also suctioned. The additional openings 26 prevent a negative pressure within the base body 12 if the suction opening 20 is sealed by the tongue or cheek of the patient, for example. Three additional openings 26 are provided in the exemplary embodiment; however, only a single additional opening 26 or even more than three additional openings 26 are also conceivable.

Profile members 38, which provide for a safe grip of the mirror suction device 10 and prevent the fingers of the attending dentist from slipping, are discernible on the outer surface 16 of the base body 12.

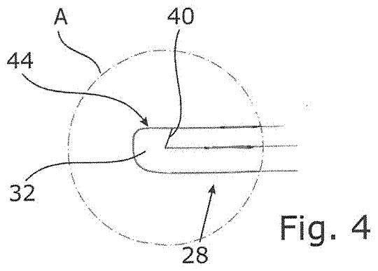

FIGS. 3 and 4 show a mirror-accommodating portion 48 into which the mirror 22 is inserted in the assembled state. An inner wall 30 of the opening 28 surrounds the mirror 22 and abuts against an outer wall of the mirror 22 at least in some portions. The opening 28 narrows starting from an underside 42 of the base body in the direction towards the suction opening 20 and forms a retaining shoulder 40.

In cross section, the mirror 22 may also be configured to be approximately trape-zoidal zoidal at least in some portions, so that its diameter increases at least in some portions, starting from the mirror surface 24 in the direction towards an underside of the base body 42. This shape simplifies the insertion or snap-in attachment in the mirror-accommodating portion 48. In this case, the retaining shoulder 40 rests against the outer circumference, which narrows in the direction towards the mirror surface 24.

In an enlarged illustration of the region A from FIG. 4, FIG. 4 illustrates that the upper retaining shoulder 40 surrounds the entire outer wall 36 of the mirror 22 and seals a space next to and below the mirror 22. The seal is improved by means of a bias of the upper retaining shoulder 40. This means that the mirror 22, when it is inserted into the first base body part 32, is pressed against the upper retaining shoulder 40, and the latter is minimally compressed or elastically deformed. Advantageously, the back of the mirror has a smaller diameter in cross section than the outermost diameter of the mirror-accommodating portion 48 in the region of the retaining shoulder 40.

The disclosure is not limited to the exemplary embodiments described, but also includes all embodiments acting in an equivalent way. The above-described variant of an embodiment is to be understood only as an example, and not as limiting. It is also possible to combine the technical features shown in any technically meaningful manner.

1. A dental mirror suction device for suctioning liquids and particles from an oral cavity of a patient, the dental mirror suction device comprising: a tubular hollow base body having an inner surface, an outer surface, a longitudinal axis, a connection opening for a hose, and a suction opening, wherein the inner surface has a mirror, which is viewable at least in some portions through the suction opening, with a mirror surface and a mirror back,
wherein the base body has a single-piece configuration and has in the inner surface, in the region of the suction opening, a mirror-accommodating portion, which is configured as a depression and in which the mirror is positively retained.

2. The dental mirror suction device according to claim 1,
wherein
the mirror-accommodating portion, starting from an underside of the base body, narrows in the direction towards the suction opening, and an inner wall of the mirror-accommodating portion forms a retaining shoulder contacting an upper face of the mirror and retaining the mirror in the mirror-accommodating portion.

3. The dental mirror suction device according to claim 2, wherein the retaining shoulder contacts the upper face over the entire circumference.

4. The dental mirror suction device according to claim 1, wherein the mirror is formed in a circular manner.

5. The dental mirror suction device according to claim 1, wherein the mirror is retained without adhesive.

6. The dental mirror suction device according to claim 1, wherein the base body is made from plastic.

7. A method for producing a dental mirror suction device for suctioning liquids and particles from an oral cavity of a patient, with a hollow base body having an outer surface, an inner surface, a longitudinal axis and a suction port, wherein the inner surface has a mirror viewable in at least some portions through the suction port, the method including the following steps:
manufacturing a base body with a mirror-accommodating portion, which is configured as a depression and has a substantially peripherally extending, elastic upper retaining shoulder, for the mirror, and
inserting the mirror from above by pressing it into the mirror-accommodating portion in such a way that the upper retaining shoulder, due to its elasticity, is first pressed outwards by the mirror and then contacts an upper face of the mirror and retains the mirror in the mirror-accommodating portion.

8. The method according to claim 7, wherein the mirror is configured in a manner tapering conically in cross section, wherein the cross section decreases from the mirror surface in the direction towards the back of the mirror.
